(19) United States
(12) Patent Application Publication
(10) Pub. No.: US 2024/0358725 A1
(43) Pub. Date: Oct. 31, 2024

(54) INHALANT CANNABIDIOL (CBD) IN THE TREATMENT OF GLIOBLASTOMA

(71) Applicant: AUGUSTA UNIVERSITY RESEARCH INSTITUTE, INC., Augusta, GA (US)

(72) Inventors: Babak BABAN, Augusta, GA (US); Jack YU, Augusta, GA (US)

(73) Assignee: AUGUSTA UNIVERSITY RESEARCH INSTITUTE, INC., Augusta, GA (US)

(21) Appl. No.: 18/682,693

(22) PCT Filed: Aug. 10, 2022
(86) PCT No.: PCT/US2022/039957

§ 371 Date: Feb. 9, 2024

Related U.S. Application Data
(60) Provisional application No. 63/231,589, filed on Aug. 10, 2021.

(51) Int. Cl. A61K 31/00; A61K 9/00; A61P 35/00

(52) U.S. Cl. CPC A61K 31/658; A61K 9/008; A61P 35/00

(57) ABSTRACT

A method of reducing tumor size in a subject in need thereof, comprising administering to the subject an effective amount of cannabidiol effective to inhibit tumor growth.

### CROSS-REFERENCE TO RELATED APPLICATIONS

The present application claims the benefit of priority to U.S. Provisional Application No. 63/231,589, filed on Aug. 10, 2021, the contents of which are incorporated by reference herein.

### STATEMENT REGARDING FEDERALLY SPONSORED RESEARCH

This invention was made with government support under ROINS110378 awarded by the National Institute of Health. The government has certain rights in the invention.

### FIELD OF THE INVENTION

One aspect of the invention is generally directed to inhalant cannabidiol (CBD) compositions and methods of their use.

### BACKGROUND OF THE INVENTION

Glioblastoma multiform (GBM), the most common malignant brain tumor, is highly invasive locally, recurs often, and has poor prognosis (Kanderi, T., et al., StatPearls Publishing, PMID: 32644380 (2021); D′Alessio, A., et al., Cancers (Basel), 11 (4): 469 (2019): Kim, J. H., et al., Am J Surg Pathol, 36 (4): 620-628 (2012)). Despite advances in cancer therapies, GBM remains incurable, with a median survival of only 15 months (Ladomersky, E., et al., Clin Cancer Res., 24 (11): 2559-2573 (2018): Chen, P. Y., et al., Front Immunol., 9 (10): 2395 (2019)). Current standards of care for GBM, including surgery, radiotherapy, and chemotherapy, produce only limited responses. Therefore, an urgent need exists for the development of novel, more effective alternative therapeutic modalities in the treatment of GBM.

Tumor microenvironment (TME), a complex network of many cell types, blood vessels, lymphatics, and immune signaling, and extracellular matrices, plays significant and integral role in the progression of cancer (Araya, R. E., et al., Methods Enzymol., 632:309-337 (2020): Roma-Rodrigues, C., et al., Int J Mol Sci., 20 (4): 840 (2019): Schiffer, D., et al., Cancers (Basel), 11 (1): 5 (2018)). The interplay between angiogenic and immunogenic compartments within TME is of fundamental importance to tumor survival and, thus poor patient outcomes (Gargini, R., et al., Cancers (Basel), 12 (6): 1622 (2020)). Therefore, targeting angiogenesis and immunologic components may alter the ecosystem of TME with beneficial outcomes for patients with GBM. Recent studies have suggested a central role for apelin, an inotropic peptide with proangiogenic features during the progression of GBM (Ishimaru, Y., et al., Sci Rep, 7 (1): 15062 (2017): Helker, C. S., et al., Elife, 9: e55589 (2020); Mastrella, G., et al., Cancer Res, 79 (9): 2298-2313 (2019): Harford-Wright, E., et al., Brain, 140 (11): 2939-2954 (2017)). While there was a minimal level of apelin expression in normal brain tissues, however, apelin expression was significantly elevated in GBM. Inhibition of apelin have resulted in the decrease in the growth rate of GBM tumor volume.

Several studies have reported a very distinctive immune profile within TME of glioblastoma, characterized by heightened immune checkpoint signaling, accumulated suppressive myeloid cells, and a decrease in effector lymphoid cells. There is a particularly low frequency of cytotoxic T cells (Antunes, A. R. P., et al., Elife, 9: e52176 (2020)). The reciprocal communication between immune compartment and non-immune components of TME (e.g., tumor cells, endothelial cells, vascular system) in GBM not only determines the status of the immune profile of TME, but also affects the vascularization, angiogenesis, and ultimately the longevity and viability of the tumor itself (Quail, D. F., et al., Cancer Cell, 31 (3): 326-341 (2017); Hambardzumyan, D., et al., Trends Cancer, 1 (4): 252-265 (2015)). Importantly, cellular Immunity, mediated by effector T cells is the major arm of immune system against tumor antigens and cancer progression (Chen, D., et al., Cancer Biol Med. 14 (2): 121-128 (2017): Hiam-Galvez, K. J., et al., Nat Rev Cancer, 21 (6): 345-359 (2021)). Inhibiting T cell activation, particularly auto-reactive CD8+ cytotoxic T cells, is a central immunomodulatory strategy by which immune checkpoints exert their suppressive role against anti-tumor immunity within GBM (Leclerc, M., et al., Nat Commun, 10 (1): 3345 (2019): Principe, N., et al., Front Immunol, 11:584423 (2020): Yang. I., et al., J Clin Neurosci, 17 (11): 1381-1385 (2010)). As an immune checkpoint, indoleamine 2,3, dioxygenase (IDO), a rate-limiting enzyme with inhibitory effects on T cells, has emerged as a very attractive potential target in the immunotherapy of several types of cancer including GBM (Brown, Z. J., et al., Cancer Immunol Immunother, 67 (8): 1305-1315 (2018): Zhai, L., et al., Cell Mol Immunol, 15 (5): 447-457 (2018): Romani, M., et al., Front Oncol, 8:464 (2018): Zhai, L., Clin Cancer Res, 23 (21): 6650-6660 (2017)). Due to its unique potential immunomodulatory function, IDO is considered a non-conventional immune checkpoint with overarching effects on chronic inflammation, antigen presentation, and immune-tolerance for tumor ecosystem. Along with IDO, recent studies have demonstrated that P-selectin may serve as an immune checkpoint within TME, promoting tumor growth in GBM (Yeini, E., et al., Nat Commun, 12 (1): 1912 (2021): Nolo, R., et al., Oncotarget, 8 (49): 86657-86670 (2017)). P-selectin is a transmembrane protein acting as a cell adhesion molecule on the surfaces of activated endothelial cells and platelets, providing the foundation for interplay between tumor cells and cellular components of the blood (Lubor, B., Glycobiology, 28 (9): 648-655 (2018)).

Further, the phylogenetically ancient, but newly discovered members of TME are Innate Lymphoid Cells (ILCs) (Walker, J. A., et al., Nat Rev Immunol, 13 (2): 75-87 (2013): Hagerling, C., et al., Trends Cell Biol, 25 (4): 214-220 (2015): Tugues, S., et al., Semin Immunol, 41:101270 (2019)). These special lymphoid cells mirror T-helper cells but possess neither T cell receptors nor lymphoid surface markers except CD45 (Artis, D., et al., Nature, 517 (7534): 293301 (2015): Bennstein, S. B., et al., FEBSJ, (2021): Baban, B., et al., JCI Insight, 6 (1): e126766 (2021)). The role of ILCs in tumor development and cancer progression is controversial and yet to be elucidated (Ghaedi, M., et al., Cell Res, 30 (7): 562-563 (2020)). However, increasing evidence suggests a central role for ILCs (including natural killer, NK, cells) in GBM (Sedgwick, A. J., et al., Front Immunol, 11:1549 (2020)). Given the complexity and heterogeneity of glioblastoma, an alternative treatment to alter the TME by inhibiting Immune checkpoints may be a potential therapeutic modality with significant beneficial effects for patients with GBM.

Cannabidiol (CBD) is a relatively safe, non-psychoactive phytocannabinoid produced by cannabis plants. Recent work by our laboratory and others suggest a beneficial effect of CBD alone or in combination with other cannabinoids in the treatment of malignancies (Dariš, B., et al., Bosn J Basic Med Sci, 19 (1): 14-23 (2019): Alexander, A., et al., Cancer Lett, 285 (1): 6-12 (2009): Dell, D. D., et al., J Adv Pract Oncol, 12 (2): 188-201 (2021): Griffiths, C., et al., Biomolecules, 11 (5): 766 (2021): Simmerman, E., et al., J Surg Res, 235:210-215 (2019)): however, few studies have investigated the efficacy of CBD as mono-therapy and/or as an adjunct with other, conventional, anticancer medications in the treatment of GBM (Doherty, G. J., et al., Br J Cancer, 124 (8): 1341-1343 (2021): Wang, K., et al., Biomed Res Int, 2021:6612592 (2021): Volmar, M. N. M., et al., Neuro Oncol, noab095 (2021): Dumitru, C. A., et al., Front Mol Neurosci, 11:159 (2018)).

In this study, we tested for the first time, the potential effect of inhalant CBD in the progression of glioblastoma in a murine model, and whether such treatment could alter the TME of glioblastoma. Our findings demonstrated the potential of inhalant CBD in the inhibition of tumor growth with alterations of TME in glioblastoma.

### SUMMARY OF THE INVENTION

Disclosed herein are cannabinoid based compositions and methods of their use to treat or reduce tumor growth and associated symptoms. One embodiment discloses a method of reducing tumor size in a subject in need thereof, comprising administering to the subject an effective amount of cannabidiol effective to inhibit tumor growth. In another embodiment, the subject has a glioblastoma multiforme tumor.

In another embodiment, administering the effective amount of cannabidiol alters a balance between stimulating versus inhibitory forces during tumor angiogenesis within the tumor microenvironment. The alteration of the balance between stimulating versus inhibitory forces during tumor angiogenesis results in decreased expression of angiogenic factors within the tumor microenvironment. And decreased expression of angiogenic factors inhibits the tumor's growth. The angiogenic factors are selected from a group consisiting of P-selectin, apelin, and IL-8.

In another embodiment, administering the effective amount of cannabidiol alters the tumor's intra-tumor vascularization and immune profile. Administering the effective amount of cannabidiol to the tumor decreases expression of immune checkpoint signaling factors to alter the immune profile of the tumor. The immune checkpoint signaling factors are selected from a group consisting of IDO and P-selectin. The decrease in the expression of immune checkpoint signaling factors inhibits tumor growth. Also, the decrease in the expression of immune checkpoint signaling factors activates or enhances anti-tumor immunity. Anti-tumor immunity is activated or enhanced by increasing frequency of CD8+ cells and improving antigen presentation through heightened CD103 expression.

In another embodiment, administering the effective amount of cannabidiol to the tumor reduces frequencies of innate lymphoid cells (ILCs) within tumor microenvironment, thereby improving anti-tumor immunity.

Another embodiment discloses, a pharmaceutical composition comprising an effective amount of a cannabinoid to inhibit tumor growth. In one embodiment, the composition is formulated for administration through inhalation. In another embodiment, the composition is formulated for pulmonary administration. In another embodiment, the composition is formulated for nasal administration. In yet another embodiment, the composition is formulated for aerosol administration.

In one embodiment, the cannabinoid of pharamaceutical composition is selected from the group consisting of tetrahydrocannabinols (THC), delta-9-tetrahydrocannabinol and delta-8-tetrahydrocannabinol, cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV), monomethyl cannabigerol ether (CBGM), arachidonoylethanolamine (AEA), 2-arachidonoylglycerol (2-AG), 2-arachidonyl glyceryl ether (noladin ether), N-arachidonoyl dopamine (NADA), virodhamine (OAE) lysophosphatidylinositol (LPI), nabilone, rimonabant, JWH-073, CP-55940, dimethylheptylpyran, HU-210, HU-331, SR144528, WIN 55,212-2, JWH-133, levonantradol, and AM-2201 and combinations thereof. In another embodiment the cannabinoid is cannabidiol.

### BRIEF DESCRIPTION OF THE DRAWINGS

FIGS. 1A-1J show that inhalant CBD inhibits tumor growth in glioblastoma.

FIGS. 1A-1B shows optical imaging results (bioluminescence imaging after injecting luciferin) displaying the establishment and growth of glioblastoma tumor at day 8 post implantation. FIGS. IC-ID shows tumor growth was inhibited after 8 consecutive daily treatments of inhalant CBD (total of 16 days including 8 days after implantation) compared to the placebo treated group. FIG. IE is a bar graph showing the quantification of optical bioluminescence images after placebo and CBD treatment (*p>0.05). FIG. IF illustrates the treatment of mice with CBD/placebo using inhaler ApelinDx. FIGS. IG-II shows H&E staining (left panel) demonstrating significant decrease in tumor size after CBD treatment compared to placebo. The difference in tumor size between CBD and placebo treated groups (p<0.05) was visualized and quantified using ImageJ Java-based image processing program (middle and right panels). FIG. 1J shows a survival curves of animals in CBD-treated group at 16 days after orthotopic implantation of the tumor, compared to animals from placebo group ( 3/6).

FIGS. 2A-2M show that inhalant CBD alters tumor microenvironment (TME), repressing angiogenic factors in glioblastoma. Immunohistochemical staining of paraffin embedded glioblastoma tumor tissues showed inhalant CBD decreased expression of angiogenic factors: P-Selectin (FIGS. 2A-2D), apelin (FIGS. 2E-2H) and IL-8 (FIGS. 21-2L) significantly compared to the placebo treated group as quantified (*p>0.05: ** p>0.01) (FIG. 2M). All images have been obtained using bright field Zeiss (AXIO Imager M2) light microscope, magnifications of 200× and 630×.

FIGS. 3A-3M show that inhalant CBD modulates immune checkpoints within TME in glioblastoma, altering the intra-tumoral immune profile. Immunohistochemical staining of paraffin embedded GBM tumor tissues showed inhalant CBD blocked the immune checkpoint, IDO (FIGS. 3A-3D) while enhanced CD8 (FIGS. 3E-3H) and CD103 (FIGS. 31-3L) expression compared to the placebo treated group as quantified (*p>0.05: ** p>0.01) (FIG. 3M). All images have been obtained using bright field Zeiss (AXIO Imager M2) light microscope, magnifications of 200× and 630×.

FIGS. 4A-4D show that inhalant CBD decreased innate lymphoid cells frequencies in glioblastoma, regulating local proliferation and activation of ILCs within TME. Flow cytometry analysis showed that inhalant CBD was able to reduce frequencies of ILCs within TME of glioblastoma significantly (p<0.02), potentially improving anti-tumor immunity. ILCs were characterized by live gating of total tumor cells (FIG. 4A-4B) based on forward scatter/side scatter (FSC/SSC), and further gating (FIG. 4C-4D) as CD45⁺ Lineage negative (CD3ε, CD11b, CD24, CD5, CD11c, CD19, NK1.1, Gr-1, TER119 and gd TCR), followed by additional gating (FIG. 4E) as Lin-CD45+Thyl⁺. A pie chart (FIG. 4F) displays the representation of the ratio of ILCs counts of CBD treated (green 33%) vs placebo group (red 67%).

### DETAILED DESCRIPTION OF THE INVENTION

### I. Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%: in other embodiments the values may range in value either above or below the stated value in a range of approx. +/−5%: in other embodiments the values may range in value either above or below the stated value in a range of approx. +/−2%: in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “antigen” refers to a substance that is capable of stimulating an immune response. In general, two main divisions of antigens are recognized: foreign antigens (or heteroantigens) and autoantigens (or self-antigens). Foreign antigens originate from outside the body. An antigen can be a protein, peptide, polysaccharide, DNA, RNA, or small molecules coupled to carrier proteins (haptens). An autoantigen refers to an antigen that is a normal bodily constituent and against which the immune system produces autoantibodies.

As used herein, “cannabidiol (CBD)” refers to the major nonpsychotropic cannabinoid compound derived from the plant Cannabis sativa, commonly known as marijuana. CBD has been reported to have antioxidant, anti-inflammatory and neuroprotective effects, which occur independent of the canonical cannabinoid CB1 and CB2 receptors.

As used herein, “Glioblastoma” refers to an aggressive cancer occurring in the brain or spinal cord. It can result in aggressive tumors known as Glioblastoma multiforme.

As used herein, “Glioblastoma multiforme (GBM)” refers to the most common malignant brain tumor resulting from Glioblastoma. GBM is highly invasive locally, recurs often, and has poor prognosis. GBM remains incurable, with a median survival of only 15 months. Current standards of care for GBM, including surgery, radiotherapy, and chemotherapy, produce only limited responses.

As used herein, an “immune checkpoint” is a pathway in the immune system that maintains self-tolerance. Immune checkpoints also serve to prevent autoimmunity and control immune responses. TME's of glioblastoma are characterized by heightened immune checkpoint signaling.

As used herein, “indoleamine 2,3, dioxygenase (IDO)” is a rate-limiting enzyme with inhibitory effects on T cells. IDO has emerged as a very attractive potential target in the immunotherapy of several types of cancer including GBM due to its unique potential immunomodulatory function.

As used herein, “P-Selectin” is a transmembrane protein acting as a cell adhesion molecule on the surfaces of activated endothelial cells and platelets, providing the foundation for interplay between tumor cells and cellular components of the blood. Recent studies have demonstrated that P-selectin may serve as an immune checkpoint within TME, promoting tumor growth in GBM

As used herein, a “tumor” refers to an abnormal growth of tissue within the body.

As used herein, “tumor microenvironment (TME)” refers to a complex network of many cell types, blood vessels, lymphatics, and immune signaling, and extracellular matrices. TME's play significant and integral roles in the progression of cancer. The interplay between angiogenic and immunogenic compartments within TME is of fundamental importance to tumor survival in cancer patients.

### II. Altering Tumor Microenvironments of GBM

Several studies have reported a very distinctive immune profile within TME of glioblastoma, characterized by heightened immune checkpoint signaling, accumulated suppressive myeloid cells, and a decrease in effector lymphoid cells. There is a particularly low frequency of cytotoxic T cells (Roma-Rodrigues, C., et al., Int J Mol Sci., 20 (4): 840 (2019): Gargini, R., et al., Cancers (Basel), 12 (6): 1622 (2020): Antunes, A. R. P., et al., Elife, 9: e52176 (2020)). The reciprocal communication between immune compartment and non-immune components of TME (e.g., tumor cells, endothelial cells, vascular system) in GBM not only determines the status of the immune profile of TME, but also affects the vascularization, angiogenesis, and ultimately the longevity and viability of the tumor itself (Quail, D. F., et al., Cancer Cell, 31 (3): 326-341 (2017): Hambardzumyan, D., et al., Trends Cancer, 1 (4): 252-265 (2015)).

In addition to cellular heterogeneity and matrix complexity of TME in glioblastoma, the strategic location of GBM at central nervous system (CNS) within the brain blood barrier (BBB) zone has added to the intricacy and existing challenges of therapy for GBM. Data disclosed herein (and as disclosed in Khodadadi, H., et al, Cannabis and Cannabinoid Research 10.1089/can.2021.0098 (2021) herein incorporated by reference in its entirety) showed that inhalant CBD could penetrate into the BBB, blocking angiogenic factors and altering the balance between stimulating versus inhibitory forces during the tumor angiogenesis within TME, resulting in the tumor growth inhibition. The disclosure revealed for the first time, that inhalant CBD could affect apelin, IDO, P-selectin, ILC's, and IL-8 (all of which are able to influence angiogenesis and vascularization in GBM).

### A. Apelin

Apelin, a neuro-angiogenic peptide, promotes cancer metastasis through contribution to the tumor angiogenesis, promoting cancer stem cells and drug resistance (Masoumi, J., et al., Adv Med Sci, 65 (1): 202-213 (2020)).

### B. IDO

Importantly, cellular immunity, mediated by effector T cells is the major arm of immune system against tumor antigens and cancer progression (Chen, D., et al., Cancer Biol Med. 14 (2): 121-128 (2017): Hiam-Galvez, K. J., et al., Nat Rev Cancer, 21 (6): 345-359 (2021)). Inhibiting T cell activation, particularly auto-reactive CD8+ cytotoxic T cells, is a central immunomodulatory strategy by which immune checkpoints exert their suppressive role against anti-tumor immunity within GBM (Chen, P. Y., et al., Front Immunol., 9 (10): 2395 (2019): Leclerc, M., et al., Nat Commun, 10 (1): 3345 (2019): Principe, N., et al., Front Immunol, 11:584423 (2020): Yang, I., et al., J Clin Neurosci, 17 (11): 1381-1385 (2010)). As an immune checkpoint, indoleamine 2,3, dioxygenase (IDO), a rate-limiting enzyme with inhibitory effects on T cells, has emerged as a very attractive potential target in the immunotherapy of several types of cancer including GBM (Brown, Z. J., et al., Cancer Immunol Immunother, 67 (8): 1305-1315 (2018): Zhai, L., et al., Cell Mol Immunol, 15 (5): 447-457 (2018): Romani, M., et al., Front Oncol, 8:464 (2018): Zhai, L., Clin Cancer Res, 23 (21): 6650-6660 (2017)). Due to its unique potential immunomodulatory function, IDO is considered a non-conventional immune checkpoint with overarching effects on chronic inflammation, antigen presentation, and immune-tolerance for tumor ecosystem. It functions as an immune checkpoint by depletion of tryptophan, an essential amino acid, regulating T effector cells and promoting Tregs induction.

The findings herein revealed that CBD was able to reduce the IDO expression within TME. Given the previous reports indicating the significance of IDO inhibition in limiting GBM development, the potential of CBD in down-regulation of IDO displays an effective immunotherapeutic strategy in the treatment of GBM.

### C. P-Selectin

Along with IDO, recent studies have demonstrated that P-selectin may serve as an immune checkpoint within TME, promoting tumor growth in GBM (Yeini, E., et al., Nat Commun, 12 (1): 1912 (2021): Nolo, R., et al., Oncotarget, 8 (49): 86657-86670 (2017)). P-selectin is a vascular adhesion molecule contributing to the cancer development by facilitating the cancer-endothelial cells interactions, enhancement of myeloid cell recruitment and promoting crosstalk between cancer cells and platelets (Lubor, B., Glycobiology, 28 (9): 648-655 (2018): Natoni, A., et al., Front Oncol, 6:93 (2016)). Besides the traditional role of P-selectin as a cell adhesion molecule and conciliator of cellular recruitment, several recent studies have reported that P-selectin may function as an Immune checkpoint through its receptor, PSGL-1, by regulating T cells and curtailing the Immuno-inflammatory responses (Tinoco, R., et al., Trends Immunol, 38 (5): 323-335 (2017)).

A novel finding of the studies herein is the blockade of P-selectin expression in GBM after CBD treatment. By blocking P-Selectin, inhalant CBD not only interrupted the basic P-selectin functions, but it also re-structured the glioblastoma TME.

### D. IL-8

The role of IL-8 and its receptors CXCR½ in the progression of several cancers including GBM have been demonstrated (Sharma, I., J Biomed Sci, 25 (1): 62 (2018)). Accordingly, as a potent angiogenic factor, IL-8 plays a crucial role in the progression as well as invasion of GBM, altering TME in glioblastoma, and affecting the angiogenesis process in an autocrine/endocrine fashion. Regulation of NF-κB, NO (nitric oxide) signaling, as well as the inhibition of crosstalk between IL-8 and the intra-tumor IL-6/VEGF are examples of potential mechanisms by which targeting IL-8 may limit the tumor growth in GBM (Raychaudhuri, B., et al., J Neurooncol, 101 (2): 227-35 (2011): Guequén, A., et al., Front Physiol, 10:988 (2019): Luo, X., et al., Oncology, 6171-6179 (2019): Pasi, F., et al., Anticancer Res, 30 (7): 2769-2772 (2010)). While several previous studies had shown the suppressive effect of CBD on IL-8 (Anil, S. M., et al., Sci Rep, 11 (1): 1462 (2021)), the findings herein show for the first time the down-regulation of IL-8 in GBM via inhalant CBD, supporting the notion that CBD may be used as an immunotherapeutic agent in the treatment of GBM.

### E. Innate Lymphoid Cells (ILCs)

Further, the phylogenetically ancient, but newly discovered members of TME are Innate Lymphoid Cells (ILCs) (Walker, J. A., et al., Nat Rev Immunol, 13 (2): 75-87 (2013): Hagerling, C., et al., Trends Cell Biol, 25 (4): 214-220 (2015): Tugues, S., et al., Semin Immunol, 41:101270 (2019)). These special lymphoid cells mirror T-helper cells but possess neither T cell receptors nor lymphoid surface markers except CD45 (Artis, D., et al., Nature, 517 (7534): 293301 (2015): Bennstein, S. B., et al., FEBSJ, (2021): Baban, B., et al., JCI Insight, 6 (1): e126766 (2021)). The role of ILCs in tumor development and cancer progression is controversial and yet to be elucidated (Ghaedi, M., et al., Cell Res, 30 (7): 562-563 (2020)). However, increasing evidence suggests a central role for ILCs (including natural killer, NK, cells) in GBM (Sedgwick, A. J., et al., Front Immunol, 11:1549 (2020)). Several studies have reported that TME of certain cancers are enriched with ILCs compared to the scant numbers of ILCs in normal tissues and circulation (Ducimetière, L., et al., Front Immunol, 10:2895 (2019)). The findings herein are the first demonstrating the high frequencies of ILCs within TME in glioblastoma.

The studies herein show that CBD reduces the frequencies of ILCs within TME of glioblastoma. Further, ILCs are heterogenic and plastic innate cells with high capabilities in cross-talking with all components of TME (Bruchard, M., et al., Front Immunol, 10:656 (2019): An, Z., et al., Front Immunol, 10:3111 (2020)). Therefore, CBD-induced regulation of ILCs in GBM tumors caqn be considered as an effective immunotherapeutic strategy in the treatment of GBM.

### III. Cannabidiol as a Therapeutic for Altering Glioblastoma TME's

Despite advances in cancer therapies, GBM remains incurable, with a median survival of only 15 months. Current standards of care for GBM, including surgery, radiotherapy, and chemotherapy, produce only limited responses. Therefore, an urgent need exists for the development of novel, more effective alternative therapeutic modalities in the treatment of GBM.

Cannabidiol (CBD) is a relatively safe, non-psychoactive phytocannabinoid produced by cannabis plants. Recent work by our laboratory and others suggest a beneficial effect of CBD alone or in combination with other cannabinoids in the treatment of malignancies (Dariš, B., et al., Bosn J Basic Med Sci, 19 (1): 14-23 (2019): Alexander, A., et al., Cancer Lett, 285 (1): 6-12 (2009): Dell, D. D., et al., J Adv Pract Oncol, 12 (2): 188-201 (2021): Griffiths, C., et al., Biomolecules, 11 (5): 766 (2021): Simmerman, E., et al., J Surg Res, 235:210-215 (2019)): however, few studies have investigated the efficacy of CBD as mono-therapy and/or as an adjunct with other, conventional, anticancer medications in the treatment of GBM (Doherty, G. J., et al., Br J Cancer, 124 (8): 1341-1343 (2021): Wang, K., et al., Biomed Res Int, 2021:6612592 (2021): Volmar, M. N. M., et al., Neuro Oncol, noab095 (2021): Dumitru, C. A., et al., Front Mol Neurosci, 11:159 (2018)). In this application, we show for the first time, the potential effect of inhalant CBD in the progression of glioblastoma in a murine model, and whether such treatment could alter the TME of glioblastoma. The findings demonstrate the potential of inhalant CBD in the inhibition of tumor growth with alterations of TME in glioblastoma.

### A. Cannabinoid Compositions

The term “cannabinoid” as used herein may encompass a chemical compound that activates any mammalian cannabinoid receptor, for example human CB1 receptor or human CB2 receptor. The cannabinoids may be naturally occurring (such as, for example, endocannabinoids or phytocannabinoids) or they may be synthetic. Synthetic cannabinoids may include, for example, the classical cannabinoids structurally related to THC, the non-classical cannabinoids (cannabimimetics) including the aminoalkyindoles, 1,5-diarylpyrazoles, quinolines and arylsulphonoamides, and eicosanoids related to the endocannabinoids. When a cannabinoid salt is used, it may be employed in the form of a solution. The one or more cannabinoids is preferably selected from the classical cannabinoids, more preferably selected from tetrahydrocannabinols (THC), preferably delta-9-tetrahydrocannabinol and delta-8-tetrahydrocannabinol, cannabidiol (CBD), cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV and cannabigerol monomethyl ether (CBGM). CBD is a preferred cannabinoid.

Other cannabinoids suitable for use in the present invention are endocannabinoids, substances that naturally occur in the mammalian body and which activate one or more cannabinoid receptor. Preferably endocannabinoids are selected from arachidonoy lethanolamine (AEA), 2-arachidonoylglycerol (2-AG), 2-arachidonyl glyceryl ether (noladin ether), N-arachidonoyl dopamine (NADA), virodhamine (OAE) and lysophosphatidylinositol (LPI).

Synthetic cannabinoids suitable for use in the present invention include nabilone, rimonabant, JWH-073, CP-55940, dimethylheptylpyran, HU-210, HU-331, SR144528, WIN 55,212-2, JWH-133, levonantradol, and AM-2201.

### B. Intranasal Compositions

In one embodiment, the cannabidiol compositions are formulated to allow intranasal administration. Intranasal compositions may comprise an inhalable dry powder pharmaceutical formulation comprising a therapeutic agent, wherein the therapeutic agent is present as a freebase or as a mixture of a salt and a freebase. Pharmaceutical formulations disclosed herein can be formulated as suitable for airway administration, for example, nasal, intranasal, sinusoidal, peroral, and/or pulmonary administration. Typically, formulations are produced such that they have an appropriate particle size for the route, or target, of airway administration. As such, the formulations disclosed herein can be produced so as to be of defined particle size distribution.

For example, the particle size distribution for a salt form of a therapeutic agent for intranasal administration can be between about 5 μm and about 350 μm. More particularly, the salt form of the therapeutic agent can have a particle size distribution for intranasal administration between about 5μ to about 250 μm, about 10 μm to about 200 μm, about 15 μm to about 150 μm, about 20 μm to about 100 μm, about 38 μm to about 100 μm, about 53 μm to about 100, about 53 μm to about 150 μm, or about 20 μm to about 53 μm. The salt form of the therapeutic agent in the pharmaceutical compositions of the invention can a particle size distribution range for intranasal administration that is less than about 200 μm. In other embodiments, the salt form of the therapeutic agent in the pharmaceutical compositions has a particle size distribution that is less than about 150 μm, less than about 100 μm, less than about 53 μm, less than about 38 μm, less than about 20 μm, less than about 10 μm, or less than about 5 μm. The salt form of the therapeutic agent in the pharmaceutical compositions of the invention can have a particle size distribution range for intranasal administration that is greater than about 5 μm, greater than about 10 μm, greater than about 15 μm, greater than about 20 μm, greater than about 38 μm, less than about 53 μm, less than about 70 μm, greater than about 100 μm, or greater than about 150 μm.

Additionally, the salt form of the therapeutic agent in the pharmaceutical compositions of the invention can have a particle size distribution range for pulmonary administration between about 1 μm and about 10 μm. In other embodiments for pulmonary administration, particle size distribution range is between about 1 μm and about 5 μm, or about 2 μm and about 5 μm. In other embodiments, the salt form of the therapeutic agent has a mean particle size of at least 1 μm, at least 2 μm, at least 3 μm, at least 4 μm, at least 5 μm, at least 10 μm, at least 20 μm, at least 25 μm, at least 30 μm, at least 40 μm, at least 50 μm, at least 60 μm, at least 70 μm, at least 80 μm, at least 90 μm, or at least 100 μm.

In some embodiments the disclosed cannabinoid compositions include one or more cannabinoids or pharmaceutically acceptable derivatives or salts thereof, a propellant, an alcohol, and a glycol and/or glycol ether. The alcohol may be a monohydric alcohol or a polyhydric alcohol, and is preferably a monohydric alcohol. Monohydric alcohol has a lower viscosity than a glycol or glycol ether. Accordingly, the composition is able to form droplets of a smaller diameter in comparison to compositions in which the monohydric alcohol is not present. The present inventors have surprisingly found that a specific ratio of monohydric alcohol to glycol or glycol ether results in a composition with a desired combination of both long term stability (for example the composition remains as a single phase for at least a week at a temperature of 2-40° C.) and small droplet size.

### C. Pulmonary Compositions

One embodiment provides a formulation and method for treating GBM by inhalation or pulmonary administration. The diffusion characteristics of the particular drug formulation through the pulmonary tissues are chosen to obtain an efficacious concentration and an efficacious residence time in the tissue to be treated. Doses may be escalated or reduced or given more or less frequently to achieve selected blood levels. Additionally, the timing of administration and amount of the formulation is preferably controlled to optimize the therapeutic effects of the administered formulation on the tissue to be treated and/or titrate to a specific blood level.

Diffusion through the pulmonary tissues can additionally be modified by various excipients that can be added to the formulation to slow or accelerate the absorption of drugs into the pulmonary tissues. For example, the drug may be combined with surfactants such as the phospholipids, dimyristoylphosphatidyl choline, and dimyristoylphosphatidyl glycerol. The drugs may also be used in conjunction with bronchodilators that can relax the bronchial airways and allow easier entry of the antineoplastic drug to the lung. Albuterol is an example of the latter with many others known in the art. Further, the drug may be complexed with biocompatible polymers, micelle forming structures or cyclodextrins.

### D. Aerosol Compositions

Particle size for the aerosolized drug used in the present examples was measured at about 1.0-5.0 μm with a GSD less than about 2.0 for deposition within the central and peripheral compartments of the lung. As noted elsewhere herein particle sizes are selected depending on the site of desired deposition of the drug particles within the respiratory tract.

Aerosols useful in the invention include aqueous vehicles such as water or saline with or without ethanol and may contain preservatives or antimicrobial agents such as benzalkonium chloride, paraben, and the like, and/or stabilizing agents such as polyethyleneglycol.

Powders useful in the invention include formulations of the neat drug or formulations of the drug combined with excipients or carriers such as mannitol, lactose, or other sugars. The powders used herein are effectively suspended in a carrier gas for administration. Alternatively, the powder may be dispersed in a chamber containing a gas or gas mixture which is then inhaled by the patient.

### IV. Administration

Inhalation is a convenient administration route for therapeutic agents that overcomes many of the drawbacks of oral administration, such as slow drug onset and first-pass metabolism plus it can be used with patients that suffer from pulmonary conditions.

### A. Intranasal Administration

In one embodiment, the CBD compositions are delivered through intranasal administration. As described herein, intranasal administration or nose administration comprise the described compositions being administered into the mammal nostril and reaching nasal meatus or nasal cavity. For example, the compositions can be administered with nasal spray, insufflation, nasal drop, aerosol, propellant, pressurized dispersion body, aqueous aerosol, propellant, nose suspension, instillation, nasal gel, nose is with ointment and nose ointment, by means of any new or old type equipment of administration.

One embodiment provides a method of treating glioblastoma in a subject in need thereof by administering to the subject an effective amount of a composition including cannabidiol.

### B. Pulmonary Administration

In another embodiment, the cannabinoids are delivered through pulmonary administration directly to the lungs where they are efficiently absorbed into the systemic circulation, resulting in a rapid onset of therapeutic action. The rapid onset of therapeutic action achievable through the compositions and methods of the invention offers an advantage over prior cannabinoid delivery methods such as sublingual or suppository delivery, which generally involve slower systemic absorption.

Pulmonary administration by inhalation may be accomplished by means of producing liquid or powdered aerosols, for example, by using any of various devices known in the art. PCT Publication No. WO 92/16192 dated Oct. 1, 1992; PCT Publication No. WO 91/08760 dated Jun. 27, 1991; NTIS Patent Application 7-504-047 filed Apr. 3, 1990 by Roosdorp and Crystal) including but not limited to nebulizers, metered dose inhalers, and powder inhalers. Various delivery devices are commercially available and can be employed, e.g. Ultravent nebulizer (Mallinckrodt, Inc, St. Louis, Mo.); Acorn II nebulizer (Marquest Medical Products, Englewood, Colo.); Ventolin metered dose inhalers (Glaxo Inc., Research Triangle Park, N.C.): Spinhaler powder inhaler (Fisons Corp., Bedford, Mass.) or Turbohaler (Astra). Such devices typically entail the use of formulations suitable for dispensing from such a device, in which a propellant material may be present. Ultrasonic nebulizers may also be used.

As will be understood by those skilled in the art of delivering pharmaceuticals by the pulmonary route, a major criterion for the selection of a particular device for producing an aerosol is the size of the resultant aerosol particles. Smaller particles are needed if the drug particles are mainly or only intended to be delivered to the peripheral lung, i.e. the alveoli (e.g. 0.1-3 μm), while larger drug particles are needed (e.g. 3-10 μm) if delivery is only or mainly to the central pulmonary system such as the upper bronchi. Impact of particle sizes on the site of deposition within the respiratory tract is generally known to those skilled in the art.

### EXAMPLES

### Example 1: Inhalant CBD Inhibits Tumor Growth and Improved Survival Rate in GBM

### Materials and Methods

### Animals

Wild type (WT), 9-11 week old C57BL/6 mice (obtained from Jackson Laboratories, Bar Harbor, ME USA) were used to generate the orthotopic GBM model. The animals were housed in the laboratory animal facilities of the Augusta University with free access to food and water. All the experiments were performed according to the National Institutes of Health (NIH) guidelines and regulations. The Institutional Animal Care and Use Committee (IACUC) of Augusta University (protocol #2011-0062) approved all the experimental procedures.

### Tumor Cells and Orthotopic Animal Model of GBM

To generate the orthotopic GBM model in mice, syngeneic GL261 cells were used as described previously (Ali, S., et al., PLOS One, 16 (2): e0246646 (2021)). In brief, luciferase positive GL261 cells were grown in standard growth media (RPMI-1640 plus 10% FBS) and collected in serum-free media on the day of implantation. Mice were anesthetized using 3% isoflurane and maintained with 1.5%-2% isoflurane throughout all surgical procedures. After preparation and drilling a hole at 2.25 mm to the right and 1 mm posterior to the bregma, taking care not to penetrate the dura, a 10 μL Hamilton syringe with a 26G-needle containing tumor cells (30,000) in a volume of 3 μl was lowered to a depth of 4 mm and then raised to a depth of 3 mm.

During and after the injection, a careful note was made for any reflux from the injection site. The needle was withdrawn 1 mm at a time in a stepwise manner 2-3 minutes after completing the injection. The surgical hole was sealed with bone wax. Finally, the skull was swabbed with betadine before suturing the skin. Postoperative analgesia was provided with a single injection of buprenorphine (1 mg/kg sc).

Tumor growth was determined by optical imaging (bioluminescence imaging after injecting luciferin) on day 8 post-implantation. In vivo, optical images were obtained to keep track of primary tumor and metastasis development by injecting 100 IL of luciferin (dose 150 mg/kg) intraperitoneally followed by the acquisition of bioluminescence signal by spectral AmiX optical imaging system (Spectral Instruments Imaging, Inc., Tucson, AZ). The photon intensity per mm per second was determined by Aura imaging software by Spectral Instruments Imaging, LLC (version 4.0.0).

### Treatment Protocol

The animals were further subdivided to receive either inhalant CBD or placebo (10 mg/day), delivered through an inhaler (ApelinDx, TM Global Bioscience, USA). Inhalant CBD or placebo was applied to the animals every day for a period of 8 days. At day 17 post-implantation, another set of imaging was performed before all animals were sacrificed, tumor tissues were harvested for histology and Immunohistochemical analysis as well as all flow cytometry-based assays.

### Metered Dose Tincture Inhaler: Composition and Pharmacokinetics

TM Global Bioscience USA provided the metered dose tincture inhaler, ApelinDx. As depicted in FIG. IF, ApelinDx was modified by adding an extra nozzle piece to adjust to the mouse model and to further control the intake of CBD. ApelinDx contained 985 mg of broad-spectrum CBD (winterized crude hemp extract) plus 15 mg of cosolvent, surfactant, and propellant, total volume of 1000 mg (5 mg dose per spray, with 200 mL/min flow rate). For the placebo, the 985 mg of broad-spectrum CBD was replaced with 985 mg of hemp seed oil.

### Histology and Immunohistochemistry

Freshly harvested GBM tumor tissues were fixed with 10% neutral buffered formalin (HT50-1-128: Sigma), processed, and then embedded with conventional dehydrated paraffin. All subsequent procedures were performed at room temperature. Fixed paraffin-embedded tumoral tissues were cut in 4 Im sections and stained with hematoxylin and eosin (H&E) based on standard protocol of H&E staining, observed and analyzed by a bright field light Zeiss microscope. To analyze the tumor size, we cut all tumors in half from the location with longest diameter to send for histology sectioning. We then measured and quantified the area of tumor invasion by using NIH ImageJ software (version 1.53g).

Further Immunohistochemical assessment was carried out as described previously (Khodadadi, H., et al., Cannabis Cannabinoid Res, 5 (3): 197-201 (2020)). In short, all slides were rehydrated and endogenous peroxidase activity was blocked using hydrogen peroxide diluted 1:10 with distilled water for 10 min. Sections were treated with Proteinase K for 10 min and washed twice in PBS.

Next, slides were incubated with antibodies against apelin (Bioss cat #BS-2425R-A750), IL-8 (Biorbyt Cat #Orb360891), P-selectin (Biolegend Cat #148309), IDO (SantaCruz Biotechnology Cat #SC-53978 AF594), CD103 (Biolegend Cat #121415) and CD8 (BD Biosciences Cat #553032) for two hours at room temperature. Biotinylated immunoglobulins (Biogenex cat #HK340-9K) were added to all slides for 20 min. After two washes with PBS, all slides were incubated with peroxidase-conjugated streptavidin (Biogenex cat #HK330-9K) for 20 min followed by two washes in PBS.

All slides were then treated with chromogen (Cat. No. K3461: Dako) until clearly detectable color appeared. Excess chromagen was decanted and all slides were washed by distilled water. All preparations were counterstained with hematoxylin (Cat. No. 812: ANATECH Ltd.) for 3 min and mounted in an aqueous mountant (Cat. No. 13800: LERNER Laboratories) before the analysis using bright field Zeiss (AXIO Imager M2) light microscope.

### Analytical Flow Cytometry

For flow cytometry analysis, tumor tissues were placed in a tissue culture dish with 1 mL PBS+2% FCS, 2 mg/ml of collagenase type II, and 1 mg/mL of DNase type I for 30 minutes at 37° C. Samples were then sieved through a cell strainer (BD Biosciences), followed by centrifugation (252g, 5 minutes, 4° C.) to prepare single-cell suspensions. Cells then were subjected to flow cytometry analysis using a NovoCyte Quanteun and analyzed by FlowJo analytical software.

Briefly, cells were gated as Lin-CD45+ (mouse, catalog 103114, clone 30-F11) lymphocytes and a lineage cocktail of antibodies (all antibodies from BioLegend, unless otherwise noted) included FITC-conjugated anti-CD3 (mouse, catalog 100204, clone 17A2), anti-CD4 (mouse, catalog 100406, clone GK1.5), anti-CD14 (mouse, catalog 123308, clone Sal4-2), anti-CD16 (mouse, catalog 101305, clone 93), anti-CD19 (mouse, catalog 152404, clone 1D3/CD19), anti-CD8 (mouse, catalog 140404, clone 53-5.8), anti-CD15 (human/mouse, catalog 125611, clone MC-480), anti-CD20 (mouse, catalog 152108, clone SA271G2) were used for negative selection.

Subsequently, ILCls were identified as mouse (Lin-CD127+IL-1 2Rβ2+ [mouse/human, R&D Systems, catalog FAB1959P-100, clone 305719]) cells, ILC2s were identified as mouse (Lin-CD127+GATA3+) cells, and ILC3s were identified as mouse (Lin-CD127+RORγt+: mouse/human, Thermo Fisher Scientific catalog 17-6988-82, clone AFKJS-9) cells (all antibodies from BioLegend). Isotype-matched controls were analyzed to set the appropriate gates for each sample. For each marker, samples were analyzed in duplicate.

To minimize false-positive events, the number of double-positive events detected with the isotype controls was subtracted from the number of double-positive cells stained with corresponding antibodies (not isotype control). Cells expressing a specific marker were reported as a percentage of the number of gated events. A population was considered positive for a specific marker if the population exceeded a 2% isotypic control threshold.

### Statistics

For statistical analysis, Brown-Forsythe and Welch analysis of variance (ANOVA) was used to establish significance (p<0.05) among groups. For tissue quantification statistical analysis, we compared the area of expression in both placebo and CBD-treated groups by using two-way ANOVA followed by post-hoc Sidak test for multiple comparison (p<0.05). Survival between groups was compared using Kaplan-Meier analysis and the Log-rank Mantel-Cox test.

### Results:

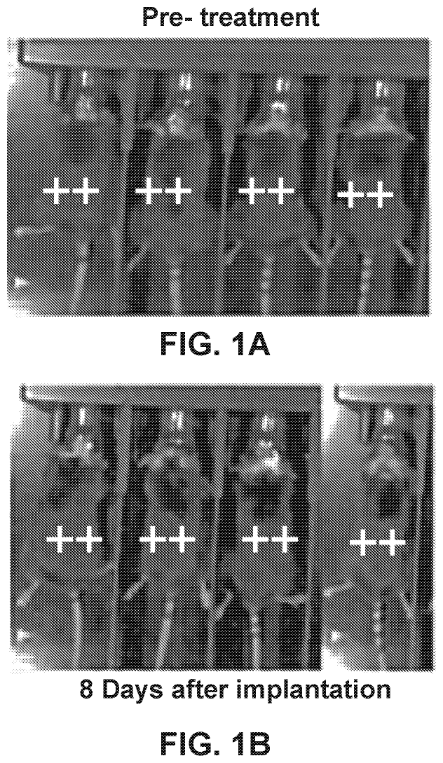

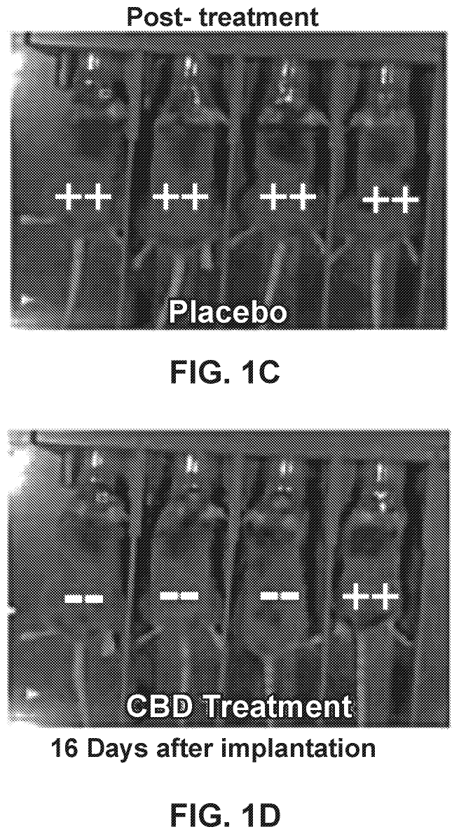

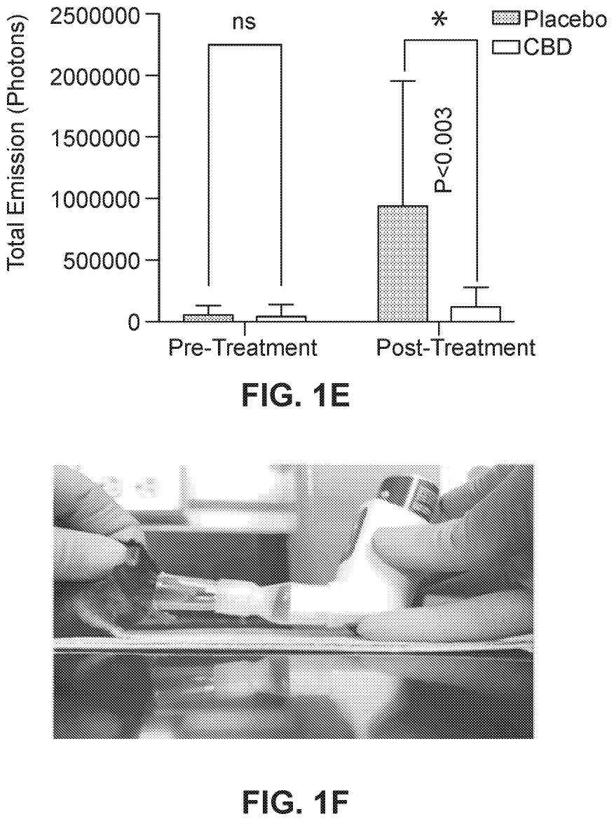

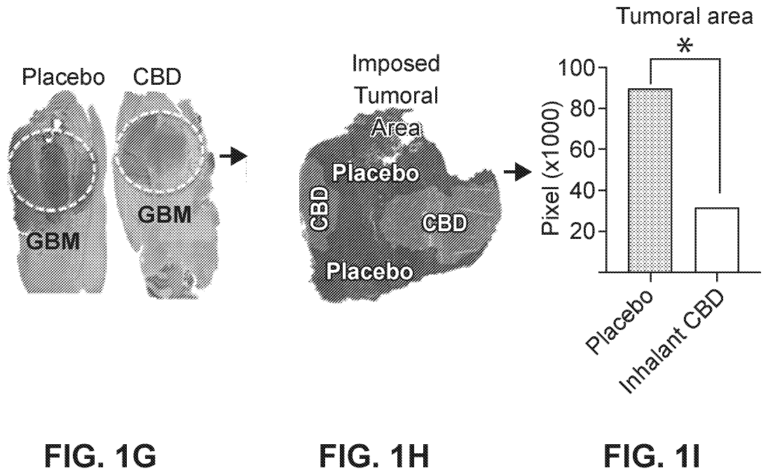

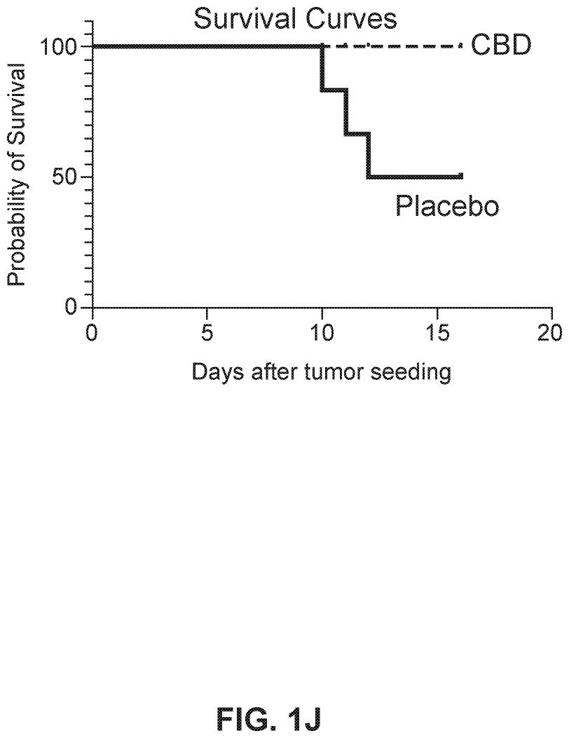

Tumor establishment was shown by optical imaging at pre-treatment stage (FIGS. 1A-1B) prior to dividing mice into two groups of placebo or CBD treated groups. As demonstrated in FIGS. 1C-ID and quantified FIG. 1E, photon intensities of optical imaging demonstrated that inhalant CBD (FIG. 1F) was able to inhibit tumor growth in GBM compared to the placebo group. Additionally, histological assessment using hematoxylin and eosin staining (H&E) revealed a significant inhibition of tumor growth (p<0.05) in CBD treated mice versus placebo control group (FIGS. 1G-1I).

Survival was evaluated (FIG. 1J), for total of eight animals (two independent of four animals per cohort), and showed that, while all animals in CBD-treated group were alive at 16 days after orthotopic implantation of the tumor, three animals (38%) from placebo group died by day 13 post-implantation, suggesting beneficial effects of CBD treatment.

### Example 2: Inhalant CBD Suppressed Angiogenic Factors within GBM Tumor

### Results:

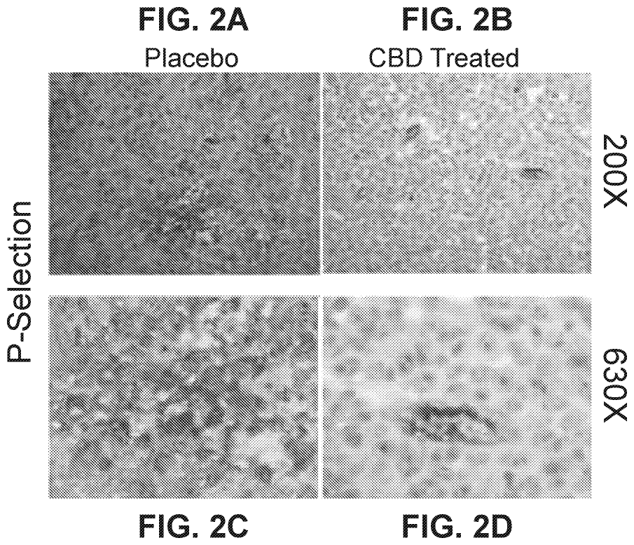

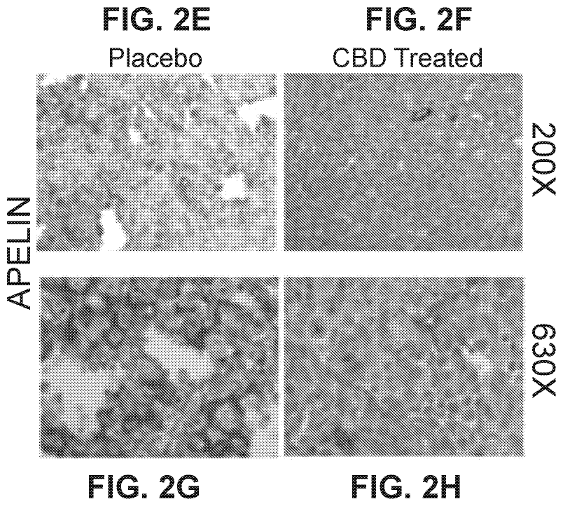

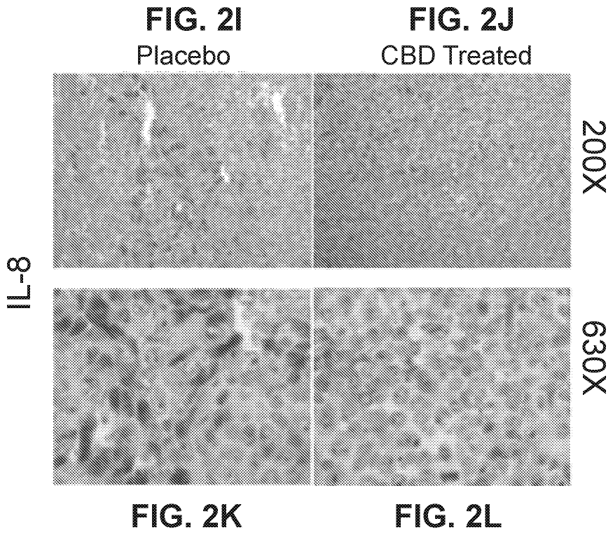

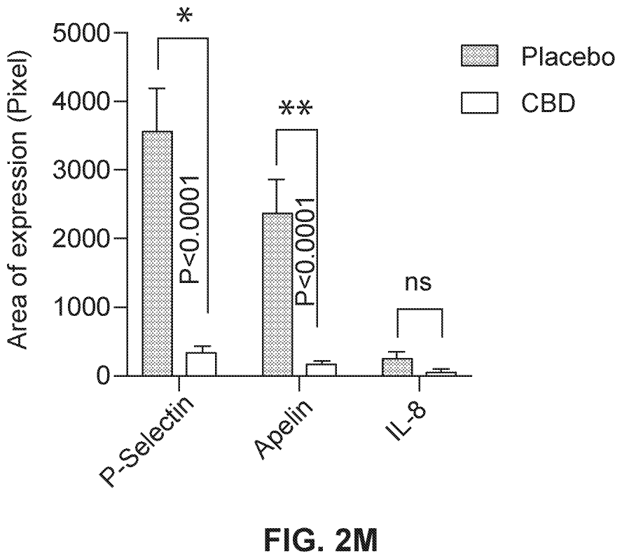

Immunohistochemistry staining showed that inhalant CBD was able to repress the expression of apelin (FIGS. 2A-2D) and P-selectin (FIGS. 2E-2H) as well as Interleukin (IL)-8 (FIGS. 21-2L), in CBD treated group compared to the placebo group as quantified (FIG. 2M). Down-regulation of these proteins can alter the TME, affecting the tumor angiogenesis negatively.

### Example 3: Inhalant CBD Blocked Immune Checkpoint Signaling, Altering the Immune Profile in GBM

### Results:

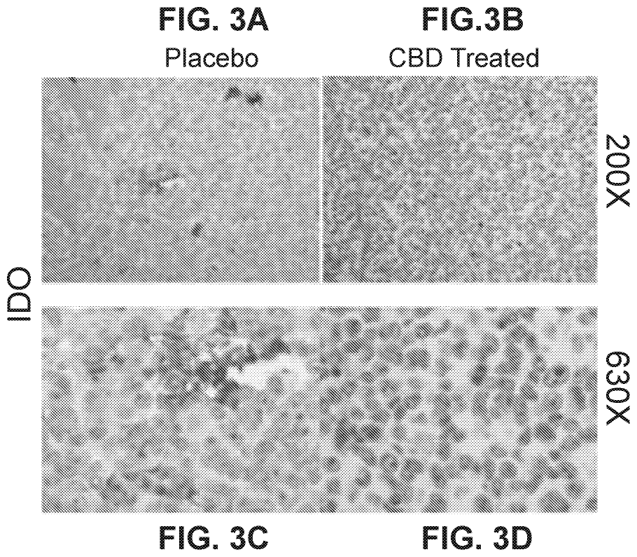

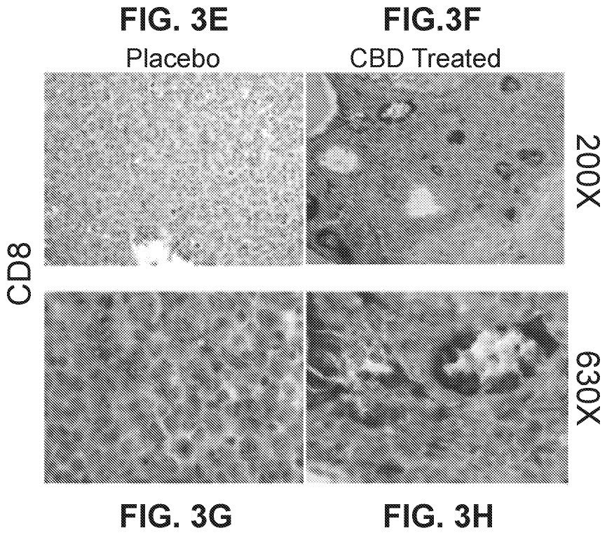

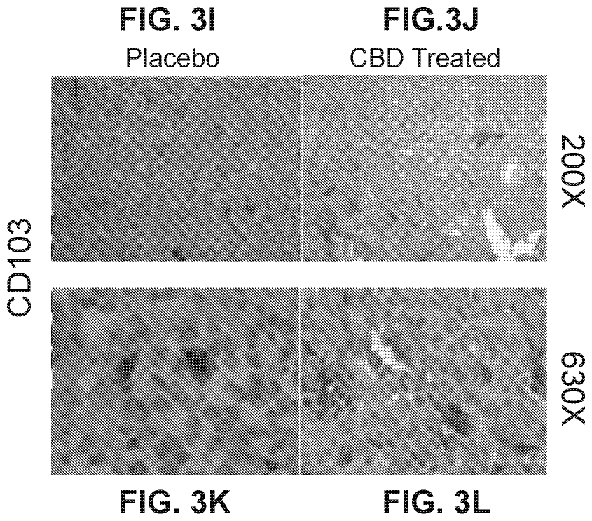

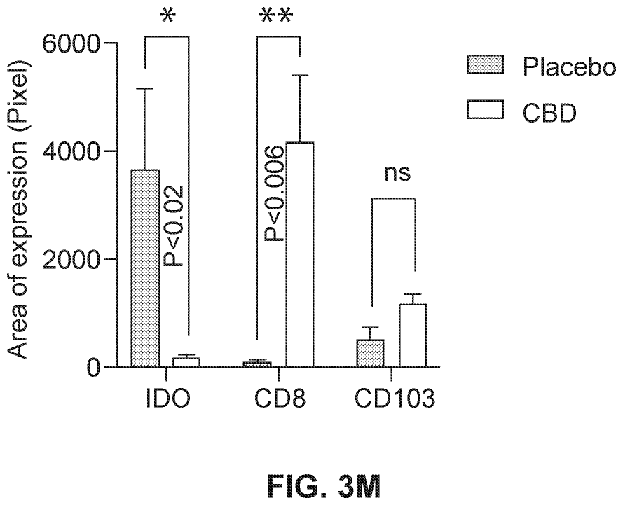

Immunohistochemistry analysis showed that inhalant CBD blocked the IDO expression (FIGS. 3A-3D) while enhanced CD8 and CD103 expression compared to placebo treated group (FIGS. 3E-3H). IDO reduction might have promoted anti-tumor immunity by increasing frequency of CD8+ cells and improving antigen presentation through heightened CD103 expression as quantified (FIGS. 31-3M). Besides IDO, CBD was able to repress the expression of P-selectin, a non-classic immune checkpoint with immunosuppressive role, enhancing antitumor immunity.

### Example 4: Inhalant CBD Regulated ILCs, Affecting Local Proliferation and Activation of ILCs within TME

### Results:

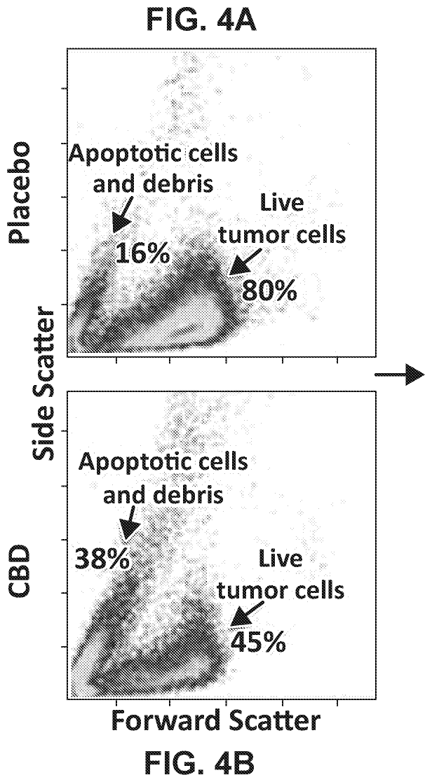

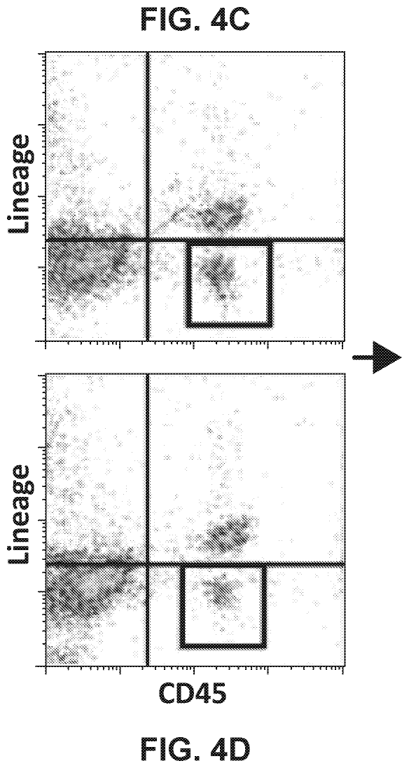

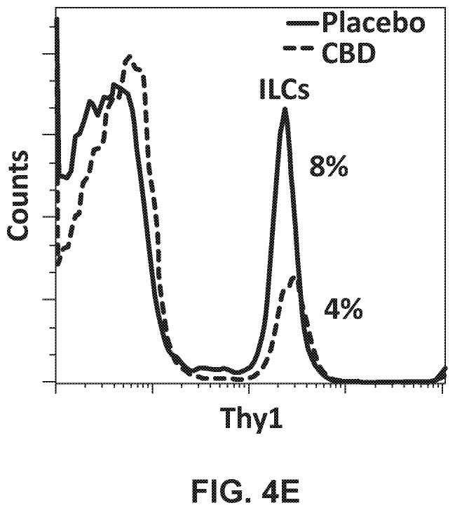

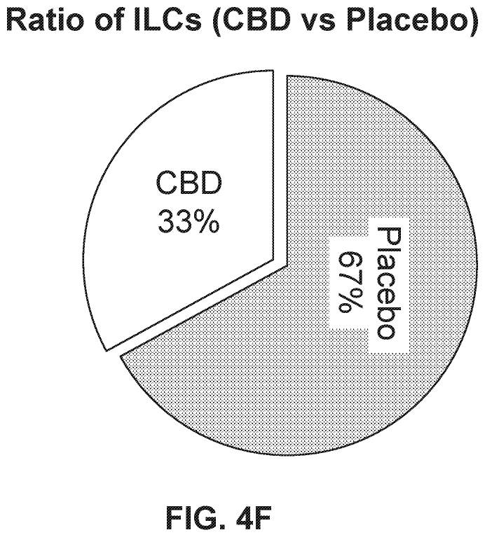

Flow cytometry analysis showed that ILCs were downregulated significantly (p<0.05) in group treated with inhalant CBD compared to the placebo group (FIG. 4A-4F). Since each cytokine produced by each class of ILCs can affect TME in a specific way, there reduction in ILCs would affect the interaction between ILCs and TME, altering the intra-tumor vascularization and immune profile.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A method of reducing tumor size in a subject in need thereof, comprising administering to the subject an effective amount of cannabidiol effective to inhibit tumor growth.

2. The method of claim 1, wherein the subject has a glioblastoma multiforme tumor.

3. The method of claim 1, wherein administering the effective amount of cannabidiol alters a balance between stimulating versus inhibitory forces during tumor angiogenesis within the tumor microenvironment.

4. The method of claim 3, wherein alteration of the balance between stimulating versus inhibitory forces during tumor angiogenesis results in decreased expression of angiogenic factors within the tumor microenvironment.

5. The method of claim 4, wherein decreased expression of angiogenic factors inhibits the tumor's growth.

6. The method of claim 4, wherein the angiogenic factors are selected from a group consisiting of P-selectin, apelin, and IL-8. Innate Lymphoid Cells (ILCs), and

7. The method of claim 1, wherein administering the effective amount of cannabidiol alters the tumor's intra-tumor vascularization and immune profile.

8. The method of claim 1, wherein administering the effective amount of cannabidiol to the tumor decreases expression of immune checkpoint signaling factors to alter the immune profile of the tumor.

9. The method of claim 8, wherein the immune checkpoint signaling factors are selected from a group consisting of IDO and P-selectin.

10. The method of claim 8, wherein the decrease in the expression of immune checkpoint signaling factors inhibits tumor growth.

11. The method of claim 8, wherein the decrease in the expression of immune checkpoint signaling factors activates or enhances anti-tumor immunity.

12. The method of claim 11, wherein anti-tumor immunity is activated or enhanced by increasing frequency of CD8+ cells and improving antigen presentation through heightened CD103 expression.

13. The method of claim 1, wherein administering the effective amount of cannabidiol to the tumor reduces frequencies of innate lymphoid cells (ILCs) within tumor microenvironment, thereby improving anti-tumor immunity.

14. A pharmaceutical composition comprising an effective amount of a cannabinoid to inhibit tumor growth.

15. The pharmaceutical composition of claim 14, wherein the composition is formulated for administration through inhalation.

16. The pharmaceutical composition of claim 14, wherein the composition is formulated for pulmonary administration.

17. The pharmaceutical composition of claim 14, wherein the composition is formulated for nasal administration.

18. The pharmaceutical composition of claim 14, wherein the composition is formulated for aerosol administration.

19. The pharmaceutical composition of claim 14, wherein the cannabinoid is selected from the group consisting of tetrahydrocannabinols (THC), delta-9-tetrahydrocannabinol and delta-8-tetrahydrocannabinol, cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV), cannabigerol monomethyl ether (CBGM), arachidonoylethanolamine (AEA), 2-arachidonoylglycerol (2-AG), 2-arachidonyl glyceryl ether (noladin ether), N-arachidonoyl dopamine (NADA), virodhamine (OAE) lysophosphatidylinositol (LPI), nabilone, rimonabant, JWH-073, CP-55940, dimethylheptylpyran, HU-210, HU-331, SR144528, WIN 55,212-2, JWH-133, levonantradol, and AM-2201 and combinations thereof.

20. The pharamaceutical composition of claim 14, wherein the cannabinoid is cannabidiol.
